(19) United States
(12) Patent Application Publication
(10) Pub. No.: US 2022/0323404 A1
(43) Pub. Date: Oct. 13, 2022

(54) LIQUID COMPOSITIONS COMPRISING TERPENES AND CANNABINOIDS

(71) Applicant: BUZZELET DEVELOPMENT AND TECHNOLOGIES LTD., OR-AKIVA (IL)

(72) Inventors: NOA RAZ, GIZO (IL); AHARON EYAL, JERUSALEM (IL)

(21) Appl. No.: 17/849,803

(22) Filed: Jun. 27, 2022

Related U.S. Application Data
(63) Continuation-in-part of application No. PCT/IB2020/062458, filed on Dec. 25, 2020.
(60) Provisional application No. 63/112,178, filed on Nov. 11, 2020.
(60) Provisional application No. 62/954,419, filed on Dec. 28, 2019.

(51) Int. Cl. A61K 31/352; A61K 31/05; A61K 31/015; A61K 31/01; A61K 31/045

(52) U.S. Cl. CPC A61K 31/352; A61K 31/05; A61K 31/015; A61K 31/01; A61K 31/045

(57) ABSTRACT

Provided is a liquid composition comprising a mixture of terpenes, at least one cannabinoid and an oil carrier. According to one embodiments, the mixture of terpenes comprises alpha-pinene, myrcene and caryophyllene and at least one additional terpene selected from the group consisting of limonene, terpinolene, linalool, geraniol, humulene, nerolidol, bisabolol and combinations thereof. According to a further embodiment, the mixture of terpenes comprises alpha-pinene, myrcene, caryophyllene, nerolidol and bisabolol, and at least one additional terpene selected from the group consisting of ocimene, limonene, linalool, and combinations thereof. According to a further embodiment, the mixture of terpenes comprises alpha-pinene, limonene, terpinolene and caryophyllene and optionally at least one additional terpene selected from the group consisting of beta-pinene, eucalyptol, geraniol and bisabolol, or combinations thereof.

### CROSS-REFERENCE TO RELATED APPLICATIONS

The present application gains priority from U.S. Provisional Patent Application Ser. No. 62/954,419 filed 28 Dec. 2019, and U.S. Provisional Patent Application Ser. No. 63/112,178 filed 11 Nov. 2020, both of which are incorporated by reference as if fully set-forth herein.

### FIELD OF THE INVENTION

The field of art to which this invention generally pertains is cannabinoid compositions, and more specifically to liquid compositions comprising terpenes, cannabinoids and an oil carrier and uses thereof in therapy.

### BACKGROUND OF THE INVENTION

Terpenes play important roles in cannabinoid-comprising products, affecting the functionality and bioavailability of the cannabinoids and the aroma of the product. Different terpenes are known to have different pharmaceutical effects in combination with cannabinoids. There is a need for compositions comprising different combinations of terpenes and cannabinoids for use in the treatment of specific indications.

### SUMMARY OF THE INVENTION

According to an aspect of some embodiments of the present invention, there is provided a liquid composition comprising alpha-pinene, myrcene and caryophyllene and at least one additional terpene selected from the group consisting of limonene, terpinolene, linalool, geraniol, humulene, nerolidol, bisabolol and combinations thereof; at least one cannabinoid; and an oil carrier.

According to a further aspect of some embodiments of the present invention, there is provided a liquid composition comprising a mixture of terpenes comprising alpha-pinene, myrcene, caryophyllene, nerolidol and bisabolol, and at least one additional terpene selected from the group consisting of ocimene, limonene, linalool, and combinations thereof; at least one cannabinoid; and an oil carrier.

According to a further aspect of some embodiments of the present invention, there is provided a mixture of terpenes consisting of alpha-pinene, limonene, terpinolene and caryophyllene, optionally comprising at least one additional terpene selected from the group consisting of beta-pinene, eucalyptol, geraniol and bisabolol, or combinations thereof; at least one cannabinoid; and an oil carrier.

### DETAILED DESCRIPTION OF THE INVENTION

The present invention, in at least some embodiments, relates to liquid compositions comprising at least one terpene, at least one cannabinoid and an oil carrier, and to uses thereof in therapy.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention.

As used herein, the term “cannabinoid” refers to a compound that affects the endocannabinoid system. Cannabinoids are agonists or antagonists to receptors in the endocannabinoid system.

As used herein, the term “THC” refers to THCa (tetrahydrocannabinolic acid) and/or to THC (tetrahydrocannabinol) unless indicated otherwise. As used herein, the term “CBD” refers to CBDa (cannabidiolic acid) and/or to CBD (cannabidiol) unless indicated otherwise. As used herein, the term “CBG” refers to CBGa (cannabigerolic acid) and/or to CBG (cannabigerol) unless indicated otherwise. As used herein, the term “CBN” refers to CBNa (Cannabinolic acid) and/or to CBN (cannabinol) unless indicated otherwise. As used herein, the term “CBC” refers to CBCa (cannabichromenic acid) and/or to CBC (Cannabichromene) unless indicated otherwise. As used herein, the term “CBL” refers to CBLa (Cannabicycol acid) and/or to CBL (Cannabicyclol) unless indicated otherwise. As used herein, the term “THCV” refers to THCVa (tetrahydrocannabivarin acid) and/or to THCV (tetrahydrocannabivarin) unless indicated otherwise. As used herein, the term “CBDV” refers to CBDVa (cannabigerovarin acid).

An oil carrier for use in the present invention may comprise an oil selected from the group consisting of vegetable oils, such as olive oil, hemp oil, soybean oil or sesame oil, or combinations thereof, as well as products thereof, such as, but not limited to, medium-chain triglycerides.

As used herein, the term “administering” includes any mode of administration, such as oral, subcutaneous, sublingual, transmucosal, parenteral, intravenous, intra-arterial, buccal, sublingual, topical, vaginal, rectal, ophthalmic, otic, nasal, inhaled, intramuscular, intraosseous, intrathecal, and transdermal, or combinations thereof. “Administering” can also include providing a different compound that when ingested or delivered as above will necessarily transform into the compound that is desired to be administered, this type of “different compound” is often being referred to as a “Prodrug”. “Administering” can also include prescribing or filling a prescription for a dosage form comprising a particular compound. “Administering” can also include providing directions to carry out a method involving a particular compound or a dosage form comprising the compound or compounds.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

As used herein, when a numerical value relating to a concentration of a terpene is preceded by the term “about”, the term “about” is intended to indicate +/−30% of that value.

As used herein, when a numerical value relating to a concentration of a cannabinoid is preceded by the term “about”, the term “about” is intended to indicate +/−20% of that value.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

According to an aspect of the present invention, there is provided a liquid composition comprising a mixture of terpenes comprising alpha-pinene, myrcene and caryophyllene; at least one cannabinoid; and an oil carrier.

According to some embodiments, said alpha-pinene is present at a concentration of from about 2000 to about 3800 ppm (such as about 2000, 2200, 2400, 2600, 2800, 3000, 3200, 3400, 3600 or 3800 ppm) by weight of the total composition; said myrcene is present at a concentration of from about 1800 to about 2800 ppm (such as about 2000, 2100, 2200, 2300, 2400, 2500, 2600, 2700 or 2800 ppm) by weight of the total composition; and said caryophyllene is present at a concentration of from about 2000 to about 3600 (such as about 2000, 2200, 2400, 2600, 2800, 3000, 3200, 3400 or 3600 ppm) by weight of the total composition.

According to some embodiments, said mixture further comprising at least one additional terpene selected from the group consisting of limonene, terpinolene, linalool, geraniol, humulene, nerolidol, bisabolol and combinations thereof.

According to some embodiments, said at least one additional terpene comprises two additional terpenes.

According to some embodiments, said two additional terpenes are selected from the group consisting of nerolidol and bisabolol; limonene and linalool; and humulene and bisabolol.

According to some embodiments, said at least one additional terpene comprises three additional terpenes.

According to some embodiments, said three additional terpenes consist of limonene, linalool and bisabolol; or geraniol, humulene and bisabolol.

According to some embodiments, said at least one additional terpene comprises five additional terpenes.

According to some embodiments, said five additional terpenes consist of limonene, terpinolene, linalool, geraniol and bisabolol.

According to some embodiments, said limonene is present at a concentration of from about 1300 to about 2300 ppm (such as about 1300, 1400, 1500, 1600, 1700, 1800, 19000, 2000, 2100, 2200 or 2300 ppm) by weight of the total composition.

According to some embodiments, said terpinolene is present at a concentration of from about 1300 ppm to about 2000 ppm (such as about 1300, 1400, 1500, 1600, 1700, 1800, 1900 or 2000 ppm) by weight of the total composition.

According to some embodiments, said linalool is present at a concentration of from about 2000 to about 2800 ppm (such as about 2000, 2100, 2200, 2300, 2400, 2500, 2600, 2700, or 2800 ppm) by weight of the total composition.

According to some embodiments, said humulene is present at a concentration of from about 1300 ppm to about 2000 ppm (such as about 1300, 1400, 1500, 1600, 1700, 1800, 1900 or 2000 ppm) by weight of the total composition.

According to some embodiments, said nerolidol is present at a concentration of about 1500 ppm by weight of the total composition.

According to some embodiments, said bisabolol is present at a concentration of from about 2800 to about 3800 ppm (such as about 2800, 3000, 3100, 3200, 3300, 3400, 3500, 3600, 3700 or 3800 ppm) by weight of the total composition.

According to some embodiments, said geraniol is present at a concentration of from about 1800 to about 2300 ppm (such as about 1800, 1900, 2000, 2100, 2200 or 2300 ppm) by weight of the total composition.

According to some embodiments, the composition comprises about 3800 ppm by weight alpha-pinene, about 2000 ppm by weight myrcene, about 3200 ppm by weight caryophyllene, about 1500 ppm by weight nerolidol and about 3800 ppm by weight bisabolol.

According to some embodiments, the composition comprises about 2800 ppm by weight alpha-pinene, about 2800 ppm by weight myrcene, about 2300 ppm by weight limonene, about 2800 ppm by weight linalool, and about 2800 ppm by weight caryophyllene.

According to some embodiments, the composition comprises about 2800 ppm by weight alpha-pinene, about 2300 ppm by weight myrcene, about 1300 ppm by weight limonene, about 2000 ppm by weight linalool, about 2800 ppm by weight caryophyllene and about 2800 ppm by weight bisabolol.

According to some embodiments, said at least one cannabinoid comprises a mixture of cannabinoids selected from the group consisting of about 20% THC and about 4% CBD; about 15% THC and about 3% CBD; about 10% THC and about 10% CBD; about 10% THC and about 2% CBD; and about 3% THC and about 15% CBD.

According to some embodiments, the composition comprises about 3600 ppm by weight alpha-pinene, about 1800 ppm by weight myrcene, about 2300 ppm by weight geraniol, about 3600 ppm by weight caryophyllene, from about 1300 ppm to about 2000 ppm by weight humulene and about 3300 ppm by weight bisabolol.

According to some embodiments, the composition comprises about 2000 ppm by weight pinene, about 1800 ppm by weight myrcene, about 2000 ppm by weight limonene, from about 1300 ppm to about 2000 ppm by weight terpinolene, about 2000 ppm by weight linalool, about 1800 ppm by weight geraniol, about 2000 ppm by weight caryophyllene, and about 2800 ppm by weight bisabolol.

According to some embodiments, said at least one cannabinoid is selected from the group consisting of about 5% THC and about 10% CBD; about 3% THC and about 15% CBD; about 1% THC and about 20% CBD; about 1% THC and about 28% CBD; and about 24% CBD.

According to an aspect of some embodiments of the present invention, there is provided a composition as disclosed herein for use in the treatment of a condition selected from the group consisting of pelvic pain, endometriosis, dysmenorrhea, muscle pain, muscle spasm, joint pain, arthritis, osteoarthrosis, abdominal pain, pain associated hormonal dysfunction, hormonal imbalance, hormonal changes and combinations thereof.

According to an aspect of some embodiments of the present invention, there is provided a method for the treatment of a condition selected from the group consisting of pelvic pain, endometriosis, dysmenorrhea, muscle pain, muscle spasm, joint pain, arthritis, osteoarthrosis, abdominal pain, pain associated hormonal dysfunction, hormonal imbalance, hormonal changes and combinations thereof, the method comprising administering to said subject a composition as disclosed herein.

According to an aspect of some embodiments of the present invention, there is provided a liquid composition comprising a mixture of terpenes comprising myrcene and caryophyllene; at least one cannabinoid and an oil carrier.

According to some embodiments, said myrcene is present at a concentration of from about 1800 to about 4500 ppm (such as about 1800, 2000, 2200, 2400, 2600, 2800, 3000, 3200, 3400, 3600, 3800, 4000, 4200, 4400, or 4500 ppm) by weight of the total composition and said caryophyllene is present at a concentration of from about 2000 to about 4500 ppm (such as about 1800, 2000, 2250, 2400, 2600, 2800, 3000, 3200, 3400, 3600, 3800, 4000, 4200, 4400 or 4500 ppm) by weight of the total composition.

According to some embodiments, said mixture further comprising at least one additional terpene selected from the group consisting of alpha-pinene, beta-pinene, ocimene, linalool, limonene, bisabolol, nerolidol and combinations thereof.

According to some embodiments, said at least one additional terpene consists of linalool.

According to some embodiments, said at least one additional terpene comprises four additional terpenes.

According to some embodiments, said four additional terpenes comprise alpha-pinene, beta-pinene, ocimene and bisabolol. According to some such embodiments, the composition further comprises linalool.

According to some embodiments, said alpha-pinene is present at a concentration of from about 2000 to about 3600 ppm (such as about 2000, 2250, 2500, 2750, 3000, 3250, 3500 or 3600 ppm) by weight of the total composition.

According to some embodiments, said beta-pinene is present at a concentration of about 2000 ppm by weight of the total composition.

According to some embodiments, said ocimene is present at a concentration of about 1300 ppm by weight of the total composition.

According to some embodiments, said linalool is present at a concentration of from about 2000 to about 4500 ppm (such as about 2000, 2250, 2500, 2800, 3000, 3200, 3400, 3600, 3800, 4000, 4200, 4400 or 4500 ppm) by weight of the total composition.

According to some embodiments, said bisabolol is present at a concentration of from about 2000 to about 3600 ppm (such as about 2000, 2250, 2500, 2750, 3000, 3250, 3500 or 3600 ppm) by weight of the total composition.

According to some embodiments, the composition comprises about 3600 ppm by weight alpha-pinene, about 2000 ppm by weight beta-pinene, about 1800 ppm by weight myrcene, about 1300 ppm by weight ocimene, about 2300 caryophyllene and about 3600 bisabolol.

According to some embodiments, the composition comprises about 4500 ppm by weight myrcene, about 4500 ppm by weight linalool, about 4500 ppm by weight caryophyllene.

According to some embodiments, the composition comprises about 2000 ppm by weight alpha-pinene, about 2000 ppm by weight beta-pinene, about 2000 ppm by weight myrcene, about 1300 ppm by weight ocimene, about 2000 ppm by weight linalool, about 2000 caryophyllene and about 2000 ppm by weight bisabolol.

According to some embodiments, said at least one cannabinoid comprises a mixture of cannabinoids selected from the group consisting of about 15% THC and about 3% CBD; about 10% THC and about 10% CBD; about 10% THC and about 2% CBD; about 5% THC and about 5% CBD; and about 3% THC and about 15% CBD.

According to an aspect of some embodiments of the present invention, there is provided the composition as disclosed herein for use in the treatment of migraine, neuropathic pain and/or headache.

According to an aspect of some embodiments of the present invention, there is provided a method for the treatment of migraine, neuropathic pain and/or headache in a subject in need thereof, comprising administering to said subject a composition as disclosed herein.

According to an embodiment, said at least one additional terpene comprises five additional terpenes.

According to an embodiment, said five additional terpenes consist of alpha-pinene, beta-pinene, ocimene, limonene and nerolidol.

According to an embodiment, said at least one additional terpene comprises four additional terpenes.

According to an embodiment, said four additional terpenes consist of alpha-pinene, beta-pinene, limonene and nerolidol.

According to an embodiment, said alpha-pinene is present at a concentration of from about 1500 to about 2000 ppm by weight of the total composition.

According to an embodiment, said beta-pinene is present at a concentration of from about 1250 to about 1500 ppm by weight of the total composition.

According to an embodiment, said myrcene is present at a concentration of from about 2250 to about 3000 ppm by weight of the total composition.

According to an embodiment, said ocimene is present at a concentration of about 750 to about 1000 ppm by weight of the total composition.

According to an embodiment, said limonene is present at a concentration of from about 1250 to about 2000 ppm by weight of the total composition.

According to an embodiment, said caryophyllene is present at a concentration of from about 2250 to about 3500 ppm by weight of the total composition.

According to an embodiment, said nerolidol is present at a concentration of from about 1500 to about 2500 ppm by weight of the total composition.

According to an embodiment, the composition comprises about 2000 ppm by weight alpha-pinene, about 1500 ppm by weight beta-pinene, about 3000 ppm by weight myrcene, about 1000 ppm by weight ocimene, about 1500 ppm by weight limonene, about 3000 by weight caryophyllene and about 2000 ppm by weight nerolidol.

According to an embodiment, the composition comprises about 2000 ppm by weight alpha-pinene, about 1500 ppm by weight beta-pinene, about 3000 ppm by weight myrcene, about 2000 ppm by weight limonene, about 3500 ppm by weight caryophyllene and about 2500 ppm by weight nerolidol.

According to an embodiment, the composition comprises about 1500 ppm by weight alpha-pinene, about 1250 ppm by weight beta-pinene, about 2250 ppm by weight myrcene, about 750 ppm by weight ocimene, about 1250 ppm by weight limonene, about 2250 by weight caryophyllene and about 1500 ppm by weight nerolidol.

According to an embodiment, the composition comprises about 1500 ppm by weight alpha-pinene, about 1500 ppm by weight beta-pinene, about 3000 ppm by weight myrcene, about 750 ppm by weight ocimene, about 1500 ppm by weight limonene, about 3000 by weight caryophyllene and about 1500 ppm by weight nerolidol.

According to an embodiment, said at least one cannabinoid comprises a mixture of cannabinoids selected from the group consisting of about 15% THC and about 3% CBD; about 10% THC and about 10% CBD; about 10% THC and about 2% CBD; about 3% THC and about 15% CBD; and about 5% TCH and about 5% CBD.

According to an aspect of some embodiments of the present invention, there is provided a composition as disclosed herein, for use in the treatment of a condition selected from the group consisting of acute pain, radicular pain and/or post-surgery pain.

According to an aspect of some embodiments of the present invention, there is provided a method for the treatment of a condition selected from the group consisting of acute pain, radicular pain and/or post-surgery pain in a subject in need thereof, comprising administering to said subject the composition of any one of claims 44 to xx. According to an aspect of some embodiments of the present invention, there is provided a liquid composition comprising a mixture of terpenes comprising geraniol, caryophyllne; at least one cannabinoid; and an oil carrier.

According to some embodiments, said geraniol is present at a concentration of from about 1800 to about 2300 ppm (such as about 1800, 1900, 2000, 2100, 2200 or 2300 ppm) by weight of the total composition.

According to an embodiment, said caryophyllene is present at a concentration of from about 2000 to about 3600 ppm by weight of the total composition.

According to an embodiment, the composition further comprises at least one additional terpene.

According to some embodiments, said at least one additional terpene is selected from the group consisting of alpha-pinene, myrcene, limonene, terpinolene, eucalyptol, linalool, humulene, nerolidol, bisabolol and combinations thereof.

According to some embodiments, said at least one additional terpene comprises four additional terpenes.

According to some embodiments, said four additional terpenes comprise alpha-pinene, myrcene, humulene and bisabolol.

According to some embodiments, said at least one additional terpene comprises five additional terpenes.

According to some embodiments, said six additional terpenes consist of limonene, terpinolene, eucalyptol, linalool and nerolidol.

According to some embodiments, said at least one additional terpene comprises six additional terpenes.

According to some embodiments, said six additional terpenes consist of alpha-pinene, myrcene, limonene, terpinolene, linaloole and bisabolol.

According to some embodiments, said alpha pinene is present at a concentration of from about 2000 to about 3600 ppm (such as about 2000, 2200, 2400, 2600, 2800, 3000, 3200, 3400 or 3600 ppm) by weight of the total composition.

According to some embodiments, said myrcene is present at a concentration of about 1800 ppm by weight of the total composition.

According to some embodiments, said limonene is present at a concentration of from about 2000 to about 3600 ppm (such as about 2000, 2200, 2400, 2600, 2800, 3000, 3200, 3400 or 3600 ppm) by weight of the total composition.

According to some embodiments, said terpinolene is present at a concentration of from about 1300 to about 2000 ppm (such as about 1300, 1400, 1500, 1600, 1700, 1800, 1900 or 2000 ppm) by weight of the total composition.

According to some embodiments, said eucalyptol is present at a concentration of from about 1300 to about 2000 ppm (such as about 1300, 1400, 1500, 1600, 1700, 1800, 1900 or 2000 ppm) by weight of the total composition.

According to some embodiments, said linalool is present at a concentration of from about 2000 to about 3600 ppm (such as about 2000, 2250, 2500, 2750, 3000, 3250, 3500 or 3600 ppm) by weight of the total composition.

According to some embodiments, said humulene is present at a concentration of from about 1300 ppm to about 2000 ppm (such as about 1300, 1400, 1500, 1600, 1700, 1800, 2900 or 2000 ppm) by weight of the total composition.

According to some embodiments, said nerolidol is present at a concentration of about 1300 ppm by weight of the total composition.

According to some embodiments, said bisabolol is present at a concentration of from about 2800 to about 3300 ppm (such as about 2800, 2900, 3000, 3100, 3200 or 3300 ppm) by weight of the total composition.

According to some embodiments, the composition comprises about 3600 ppm by weight alpha-pinene, about 1800 ppm by weight myrcene; about 2300 ppm by weight geraniol; about 3600 ppm by weight caryophyllene; from about 1300 ppm to about 2000 ppm by weight humulene; and about 3300 ppm by weight bisabolol.

According to some embodiments, the composition comprises about 2000 ppm by weight alpha-pinene; about 1800 ppm by weight myrcene; about 2000 ppm by weight limonene; from about 1300 ppm to about 2000 ppm by weight terpinolene; about 2000 ppm by weight linalool; about 1800 ppm by weight geraniol; about 2000 ppm by weight caryophyllene and about 2800 ppm by weight bisabolol.

According to some embodiments, the composition comprises about 3600 ppm by weight limonene; from about 1300 ppm to about 2000 ppm by weight terpinolene; about 1300 ppm to about 2000 ppm by weight eucalyptol; about 3600 ppm by weight linalool; about 1800 ppm by weight geraniol; about 2800 ppm by weight caryophyllene; and about 1300 ppm by weight nerolidol.

According to some embodiments, said at least one cannabinoid comprises a mixture of cannabinoids selected from the group consisting of about 15% THC and about 3% CBD; about 10% THC and about 10% CBD; about 10% THC and about 2% CBD; and about 3% THC and about 15% CBD.

According to an aspect of some embodiments of the present invention, there is provided the composition as disclosed herein for use in treating a condition selected from the group consisting of anxiety, stress, depression, irritability, lack of focus, lack of alertness and poor day function.

According to an aspect of some embodiments of the present invention, there is a method for the treatment of a condition selected from the group consisting of anxiety, stress, depression, irritability, lack of focus, lack of alertness and poor day function in a subject in need thereof, the method consisting of administering to the subject a composition as disclosed herein.

According to a further aspect of some embodiments of the present invention, there is provided a liquid composition comprising a mixture of terpenes consisting of alpha-pinene, myrcene, caryophyllene, nerolidol and bisabolol; at least one cannabinoid; and an oil carrier.

According to some embodiments, said alpha-pinene is present at a concentration of from about 1000 to about 3000 ppm (such as about 1000, 1250, 1500, 1750, 2000, 2250, 2500, 2750 or 3000 ppm) by weight of the total composition.

According to some embodiments, said myrcene is present at a concentration of from about 1800 to about 5000 ppm (such as about 1800, 2000, 2200, 2400, 2600, 2800, 3000, 3200, 3400, 3600, 3800, 4000, 4200, 4400, 4600, 4800 or 5000 ppm) by weight of the total composition.

According to some embodiments, said caryophyllene is present at a concentration of from about 1400 to about 5000 ppm (such as about 1400, 1600, 1800, 2000, 2200, 2400, 2600, 2800, 3000, 3200, 3400, 3600, 3800, 4000, 4200, 4400, 4600, 4800 or 5000 ppm) by weight of the total composition.

According to some embodiments, said nerolidol is present at a concentration of from about 1200 to about 4300 ppm (such as about 1200, 1400, 1600, 1800, 2000, 2200, 2400, 2600, 2800, 3000, 3200, 3400, 3600, 3800, 4000, 4200, or 4300 ppm) by weight of the total composition.

According to some embodiments, said bisabolol is present at a concentration of from about 1000 to about 3000 ppm (such as about 1000, 1200, 1400, 1600, 1800, 2000, 2200, 2400, 2600, 2800, or 3000 ppm) by weight of the total composition.

According to some embodiments, the composition further comprises at least one additional terpene.

According to some embodiments, said at least one additional terpene is selected from the group consisting of ocimene, limonene, linalool, and combinations thereof.

According to some embodiments said at least one additional terpene consists of ocimene.

According to some embodiments, said at least one additional terpene consists of limonene.

According to some embodiments, said at least one additional terpene consists of linalool.

According to some embodiments, said at least one additional terpene comprises two additional terpenes.

According to some embodiments, said two additional terpenes consist of ocimene and linalool.

According to some embodiments, said two additional terpenes consist of limonene and linalool. According to some embodiments, said ocimene is present at a concentration of about 1800 ppm by weight of the total composition.

According to some embodiments, said limonene is present at a concentration of from about 500 to about 1800 ppm (such as about 500, 600, 700, 800, 900, 1000, 1100, 1200, 1300, 1400 or 1500 ppm) by weight of the total composition.

According to some embodiments, said linalool is present at a concentration of from about 500 to about 3000 ppm (such as about 500, 1000, 1500, 2000, 2500 or 3000 ppm) by weight of the total composition.

According to some embodiments, the composition comprises about 2800 ppm by weight alpha-pinene; about 3600 ppm by weight myrcene; about 1400 ppm by weight caryophyllene; about 4300 ppm by weight nerolidol; and about 2800 ppm by weight bisabolol.

According to some embodiments, the composition comprises about 2500 ppm by weight alpha-pinene; about 3000 ppm by weight myrcene; about 1800 ppm by weight ocimene; about 1600 ppm by weight caryophyllene; about 2200 ppm by weight nerolidol; and about 1800 ppm by weight bisabolol.

According to some embodiments, the composition comprises about 2500 ppm by weight alpha-pinene; about 3000 ppm by weight myrcene; about 1800 ppm by weight limonene; about 1600 ppm by weight caryophyllene; about 2200 ppm by weight nerolidol; and about 1800 ppm by weight bisabolol.

According to some embodiments, the composition comprises about 2500 ppm by weight alpha-pinene; about 3000 ppm by weight myrcene; about 1800 ppm by weight linalool; about 1600 ppm by weight caryophyllene; about 2200 ppm by weight nerolidol; and about 1800 ppm by weight bisabolol.

According to some embodiments, the composition comprises about 2000 ppm by weight alpha-pinene; about 2800 ppm by weight myrcene; about 1800 ppm by weight ocimene; about 2000 ppm by weight linalool; about 1400 ppm by weight caryophyllene; about 2000 ppm by weight nerolidol; and about 2000 ppm by weight bisabolol.

According to some embodiments, the composition comprises from about 1000 to about 1500 ppm (such as about 1000, 1100, 1200, 1300, 1400 or 1500 ppm) by weight alpha-pinene; from about 1800 to about 2500 ppm (such as about 1800, 1900, 2000, 2100, 2200, 2300, 2400 or 2500 ppm) by weight myrcene; about 500 ppm by weight limonene; from about 500 to about 1500 ppm (such as about 500, 600, 700, 800, 900, 1000, 1100, 1200, 1300, 1400 or 1500 ppm) by weight linalool; from about 1700 to about 2500 ppm (such as about 1700, about 1800, 1900, 2000, 2100, 2200, 2300, 2400 or 2500 ppm) by weight caryophyllene; from about 1200 to about 2000 ppm (such as about 1200, 1300, 1400, 1500, 1600, 1700, 1800, 1900 or 2000 ppm) by weight nerolidol; and about 1000 to about 1500 ppm (such as about 1000, 1100, 1200, 1300, 1400 or 1500 ppm) by weight bisabolol.

According to some embodiments, the composition comprises from about 2000 to about 3000 ppm (such as about 2000, 2100, 2200, 2300, 2400, 2500, 2600, 2700, 2800, 2900 or 3000 ppm) by weight alpha-pinene; from about 3600 to about 5000 ppm (such as about 3600, 3800, 4000, 4200, 4400, 4600, 4800 or 5000 ppm) by weight myrcene; about 1000 ppm by weight limonene; from about 1000 to about 3000 ppm (such as about 1000, 1200, 1400, 1600, 1800, 2000, 2200, 2400, 2600, 2800 or 3000 ppm) by weight linalool; from about 3400 to about 5000 ppm (such as about 3400, 3600, 3800, 4000, 4200, 4400, 4600, 4800 or 5000 ppm) by weight caryophyllene; from about 2400 to about 3000 ppm (such as about 2400, 2500, 2600, 2700, 2800, 2900 or 3000 ppm) by weight nerolidol; and about 2000 to about 3000 ppm by weight bisabolol.

According to some embodiments, said at least one cannabinoid is selected from the group consisting of about 20% THC and 4% CBD; 15% THC and about 3% CBD; about 10% THC and about 10% CBD; about 10% THC and about 2% CBD; about 5% THC and about 5% CBD; about 3% THC and about 15% CBD; about 1% THC and about 20% CBD; about 24% CBD; or about 28% CBD.

According to an aspect of some embodiments of the present invention, the composition disclosed herein is for use in treating insomnia and/or sleep disorders.

According to an aspect of some embodiments of the present invention, there is provided a method for treating insomnia and/or sleep disorders in a subject in need thereof, comprising administering to said subject a composition as disclosed herein.

According to an aspect of some embodiments of the present invention, there is provided a liquid composition comprising a mixture of terpenes consisting of alpha-pinene, limonene, terpinolene and caryophyllene; at least one cannabinoid; and an oil carrier.

According to an embodiment, said alpha-pinene is present at a concentration of from about 1000 to about 3000 ppm by weight of the total composition.

According to an embodiment, said limonene is present at a concentration of from about 1500 to about 2500 ppm by weight of the total composition.

According to an embodiment, said terpinolene is present at a concentration of from about 1500 to about 2500 ppm by weight of the total composition.

According to an embodiment, said caryophyllene is present at a concentration of from about 1500 to about 3000 ppm by weight of the total composition.

According to an embodiment, the composition further comprises at least one additional terpene. According to some such embodiments, said at least one additional terpene is selected from the group consisting of beta-pinene, eucalyptol, geraniol, bisabolol and combinations thereof.

According to an embodiment, said at least one additional terpene consists of beta-pinene. According to an embodiment, said at least one additional terpene consists of eucalyptol. According to an embodiment, said at least one additional terpene consists of geraniol. According to an embodiment, said at least one additional terpene consists of bisabolol.

According to an embodiment, said at least one additional terpene comprises two additional terpenes.

According to an embodiment, said two additional terpenes consist of beta-pinene and geraniol.

According to an embodiment, said two additional terpenes consist of beta-pinene and eucalyptol.

According to an embodiment, said two additional terpenes consist of eucalyptol and geraniol.

According to an embodiment, said beta-pinene is present at a concentration of about 1000 ppm to about 2500 ppm by weight of the total composition.

According to an embodiment, said eucalyptol is present at a concentration of from about 1500 to about 2500 ppm by weight of the total composition.

According to an embodiment, said geraniol is present at a concentration of from about 1500 to about 2500 ppm by weight of the total composition.

According to an embodiment, the composition comprises about 1000 to about 3000 ppm by weight alpha-pinene; about 1500 to about 2500 ppm by weight limonene; about 1500 to about 2500 terpinolene; and about 1500 to about 3000 ppm by weight caryophyllene.

According to an embodiment, the composition further comprises from about 1000 to about 2000 ppm by weight beta-pinene.

According to an embodiment, the composition further comprises from about 1500 to about 2500 ppm by weight eucalyptol.

According to an embodiment, the composition further comprises from about 1500 to about 2500 ppm by weight geraniol.

According to an embodiment, said at least one cannabinoid is selected from the group consisting of about 20% THC and 4% CBD; 15% THC and about 3% CBD; about 10% THC and about 10% CBD; about 10% THC and about 2% CBD; about 5% THC and about 5% CBD; about 3% THC and about 15% CBD; about 1% THC and about 20% CBD; about 24% CBD; or about 28% CBD.

According to an embodiment, the composition is for use in day time.

According to an embodiment, the composition is for use in treating difficult in focus and for improving alertness.

According to an embodiment, the composition is for use in day time in treating pain, anxiety, depression, irritability, impaired focus, impaired alertness and combination thereof.

According to an aspect of some embodiments of the present invention, there is provided a method for use during day time in treating pain, anxiety, depression, irritability, impaired focus, impaired alertness and combination thereof in a subject in need thereof, comprising administering to said subject the composition as disclosed herein.

According to an aspect of some embodiments of the present invention, there is provided a liquid composition comprising a mixture of terpenes comprising alpha-pinene, myrcene and caryophyllene and at least one additional terpene selected from the group consisting of limonene, terpinolene, linalool, geraniol, humulene, nerolidol, bisabolol and combinations thereof at least one cannabinoid; and an oil carrier.

According to an embodiment, said alpha-pinene is present at a concentration of from about 2000 to about 3800 ppm by weight of the total composition; said myrcene is present at a concentration of from about 1800 to about 2800 ppm by weight of the total composition; and said caryophyllene is present at a concentration of from about 2000 to about 3600 ppm by weight of the total composition; wherein said limonene, when present, is at a concentration of from about 1300 to about 2300 ppm by weight of the total composition; said terpinolene, when present, is at a concentration of from about 1300 ppm to about 2000 ppm by weight of the total composition; said linalool, when present, is at a concentration of from about 2000 to about 2800 ppm by weight of the total composition; said humulene, when present, is at a concentration of from about 1300 ppm to about 2000 ppm by weight of the total composition; said nerolidol, when present, is at a concentration of about 1500 ppm by weight of the total composition; wherein said bisabolol, when present is at a concentration of from about 2800 to about 3800 ppm by weight of the total composition; and said geraniol, when present, is at a concentration of from about 1800 to about 2300 ppm by weight of the total composition.

According to an embodiment, the at least one additional terpene comprises two additional terpenes, optionally selected from the group consisting of nerolidol and bisabolol; limonene and linalool; and humulene and bisabolol.

According to an embodiment, the at least one additional terpene comprises three additional terpenes, optionally selected from the group consisting of limonene, linalool and bisabolol; or geraniol, humulene and bisabolol.

According to an embodiment, the at least one additional terpene comprises five additional terpenes, optionally consisting of limonene, terpinolene, linalool, geraniol and bisabolol.

According to an embodiment, the composition comprises about 3800 ppm by weight alpha-pinene, about 2000 ppm by weight myrcene, about 3200 ppm by weight caryophyllene, about 1500 ppm by weight nerolidol and about 3800 ppm by weight bisabolol.

According to an embodiment, the composition comprises about 2800 ppm by weight alpha-pinene, about 2800 ppm by weight myrcene, about 2300 ppm by weight limonene, about 2800 ppm by weight linalool, and about 2800 ppm by weight caryophyllene.

According to an embodiment, the composition comprises about 2800 ppm by weight alpha-pinene, about 2300 ppm by weight myrcene, about 1300 ppm by weight limonene, about 2000 ppm by weight linalool, about 2800 ppm by weight caryophyllene and about 2800 ppm by weight bisabolol.

According to an embodiment, the composition comprises about 3600 ppm by weight alpha-pinene, about 1800 ppm by weight myrcene, about 2300 ppm by weight geraniol, about 3600 ppm by weight caryophyllene, from about 1300 ppm to about 2000 ppm by weight humulene and about 3300 ppm by weight bisabolol.

According to an aspect of some embodiments of the present invention, there is provided a liquid composition comprising a mixture of terpenes comprising alpha-pinene, myrcene, caryophyllene, nerolidol and bisabolol, and at least one additional terpene selected from the group consisting of ocimene, limonene, linalool, and combinations thereof; at least one cannabinoid; and an oil carrier.

According to an embodiment, said alpha-pinene is present at a concentration of from about 1000 to about 3000 ppm by weight of the total composition; said myrcene is present at a concentration of from about 1800 to about 5000 ppm by weight of the total composition; said caryophyllene is present at a concentration of from about 1400 to about 5000 ppm by weight of the total composition; said nerolidol is present at a concentration of from about 1200 to about 4300 ppm by weight of the total composition; said bisabolol is present at a concentration of from about 1000 to about 3000 ppm by weight of the total composition; said ocimene, when present, is at a concentration of about 1800 ppm by weight of the total composition; said limonene, when present, is at a concentration of from about 500 to about 1800 ppm by weight of the total composition.

According to an embodiment, said at least one additional terpene comprises two additional terpenes, optionally consisting of ocimene and linalool; or limonene and linalool.

According to an embodiment, the composition comprises about 2800 ppm by weight alpha-pinene; about 3600 ppm by weight myrcene; about 1400 ppm by weight caryophyllene; about 4300 ppm by weight nerolidol; and about 2800 ppm by weight bisabolol.

According to an embodiment, the composition comprises about 2500 ppm by weight alpha-pinene; about 3000 ppm by weight myrcene; about 1800 ppm by weight ocimene; about 1600 ppm by weight caryophyllene; about 2200 ppm by weight nerolidol; and about 1800 ppm by weight bisabolol.

According to an embodiment, the composition comprises about 2500 ppm by weight alpha-pinene; about 3000 ppm by weight myrcene; about 1800 ppm by weight linalool; about 1600 ppm by weight caryophyllene; about 2200 ppm by weight nerolidol; and about 1800 ppm by weight bisabolol.

According to an embodiment, the composition comprises about 2000 ppm by weight alpha-pinene; about 2800 ppm by weight myrcene; about 1800 ppm by weight ocimene; about 2000 ppm by weight linalool; about 1400 ppm by weight caryophyllene; about 2000 ppm by weight nerolidol; and about 2000 ppm by weight bisabolol.

According to an embodiment, the composition comprises from about 1000 to about 1500 ppm by weight alpha-pinene; from about 1800 to about 2500 ppm by weight myrcene; about 500 ppm by weight limonene; from about 500 to about 1500 ppm by weight linalool; from about 1700 to about 2500 ppm by weight caryophyllene; from about 1200 to about 2000 ppm by weight nerolidol; and about 1000 to about 1500 ppm by weight bisabolol.

According to an aspect of some embodiments of the present invention, there is provided a liquid composition comprising a mixture of terpenes consisting of alpha-pinene, limonene, terpinolene and caryophyllene, optionally comprising at least one additional terpene selected from the group consisting of beta-pinene, eucalyptol, geraniol and bisabolol, or combinations thereof; at least one cannabinoid; and an oil carrier.

According to an embodiment, said alpha-pinene is present at a concentration of from about 1000 to about 3000 ppm by weight of the total composition; said limonene is present at a concentration of from about 1500 to about 2500 ppm by weight of the total composition; said terpinolene is present at a concentration of from about 1500 to about 2500 ppm by weight of the total composition; said caryophyllene is present at a concentration of from about 1500 to about 3000 ppm by weight of the total composition; said beta-pinene, when present, is at a concentration of about 1000 ppm to about 2500 ppm by weight of the total composition; said eucalyptol, when present, is at a concentration of from about 1500 to about 2500 ppm by weight of the total composition; and said geraniol, when present, is at a concentration of from about 1500 to about 2500 ppm by weight of the total composition.

According to an embodiment, the composition comprises about 1000 to about 3000 ppm by weight alpha-pinene; about 1500 to about 2500 ppm by weight limonene; about 1500 to about 2500 terpinolene; and about 1500 to about 3000 ppm by weight caryophyllene.

### EXAMPLES

The tables below present exemplary compositions comprising specific combinations of terpenes and cannabinoids at various concentrations for use in the treatment of specific indications.

T/C concentrations represent tetrahydrocannabinol (T) to cannabidiol (C) concentration as percentage weight. For example T15/C3 refers to a cannabinoid mixture comprising 15 wt % tetrahydrocannabinol and 3 wt % cannabidiol of the total composition.

Values shown in the table are terpene concentrations in parts per million (ppm) by weight.

### Example 1: Liquid Compositions for Treatment of a Condition Selected from the Group Consisting of Pelvic Pain, Endometriosis, Dysmenorrhea, Muscle Pain, Muscle Spasm, Joint Pain and Combinations Thereof

TABLE 1

| | Cannabinoids content (wt %) | | | | |
|---|---|---|---|---|---|
| | T15/C3, T10/C10, T10/C2, T3/C15 | | | T5/C10, T3/C15, T1/C20, T0/C24, T1/C28 | |
| Terpenes | | | | | |
| (ppm wt) | | | | | |
| α-pinene | 3800 | 2800 | 2800 | 3600 | 2000 |
| Myrcene | 2000 | 2800 | 2300 | 1800 | 1800 |
| Limonene | | 2300 | 1300 | | 2000 |
| Terpinolene | | | | | 1300-2000 |
| Linalool | | 2800 | 2000 | | 2000 |
| Geraniol | | | | 2300 | 1800 |
| caryophyllene | 3200 | 2800 | 2800 | 3600 | 2000 |
| Humulene | | | | 1300-2000 | |
| Nerolidol | 1500 | | | | |
| Bisabolol | 3800 | | 2800 | 3000 | 2800 |

### Example 2: Liquid Compositions for Treatment of a Condition Selected from the Group Consisting of Migraine, Neuropathic Pain and/or Headache

TABLE 2

| | Cannabinoids content (wt %) | | |
|---|---|---|---|
| | T15/C3, T10/C10, T10/C2, T3/C15 | | |
| Terpenes | | | |
| (ppm wt) | | | |
| α-pinene | 3600 | | 2000 |
| β-pinene | 2000 | | 2000 |
| Myrcene | 1800 | 4500 | 2000 |
| Ocimene | 1300 | | 1300 |
| Linalool | | 4500 | 2000 |
| caryophyllene | 2300 | 4400 | 2000 |
| Bisabolol | 3600 | | 2000 |

### Example 3: Liquid Compositions for Treatment of a Condition Selected from the Group Consisting of Acute Pain, Radicular Pain and/or Post-Surgery Pain

TABLE 3

| | Cannabinoids content (wt %) | | | |
|---|---|---|---|---|
| | T15/C3, T10/C10, T10/C2, T3/C15, T5C5 | | T5C5 | |
| Terpenes | | | | |
| (ppm wt) | | | | |
| α-pinene | 2000 | 2000 | 1500 | 1500 |
| β-pinene | 1500 | 1500 | 1250 | 1500 |
| Myrcene | 3000 | 3000 | 2250 | 3000 |
| Ocimene | 1000 | | 750 | 750 |
| Limonene | 1500 | 2000 | 1250 | 1500 |
| caryophyllene | 3000 | 3500 | 2250 | 3000 |
| nerolidol | 2000 | 2500 | 1500 | 1500 |

### Example 4: Liquid Compositions for Treatment of a Condition Selected from the Group Consisting of Anxiety, Stress, Depression, Irritability, Lack of Focus, Lack of Alertness, Poor Day Function and Combinations Thereof

TABLE 4

| | Cannabinoids content (wt %) | | |
|---|---|---|---|
| | T15/C3, T10/C10, T10/C2, T3/C15 | | |
| Terpenes | | | |
| (ppm wt) | | | |
| α-pinene | 3600 | 2000 | |
| Myrcene | 1800 | 1800 | |
| Limonene | | 2000 | 3600 |
| Terpinolene | | 1300-2000 | 1300-2000 |
| Eucalyptol | | | 1300-2000 |
| Linalool | | 2000 | 3600 |
| Geraniol | 2300 | 1800 | 1800 |
| caryophyllene | 3600 | 2000 | 2800 |
| Humulene | 1300-2000 | | |
| Nerolidol | | | 1300 |
| Bisabolol | 3300 | 2800 | |

### Example 5: Liquid Compositions for Treatment of a Condition Selected from the Group Consisting of Insomnia and/or Sleep Disorders

TABLE 5

| | Cannabinoids content (wt %) | | | | | | |
|---|---|---|---|---|---|---|---|
| Terpenes | T20/C4, T15/C3, T10/C2, T10/C10, T5/C5, | | | | | | |
| (ppm wt) | T3/C15, T1/C20, T0/C24, T0/C28 | | | | | | |
| α-pinene | 2800 | 2500 | 2500 | 2500 | 2000 | 1000-1500 | 2000-3000 |
| Myrcene | 3600 | 3000 | 3000 | 3000 | 2800 | 1800-2500 | 3600-5000 |
| Ocimene | | 1800 | | | 1800 | | |
| Limonene | | | 1800 | | | 500 | 1000 |
| Linalool | | | | 1800 | 2000 | 500-1500 | 1000-3000 |
| caryo- | 1400 | 1600 | 1600 | 1600 | 1400 | 1700-2500 | 3400-5000 |
| phyllene | | | | | | | |
| Nerolidol | 4300 | 2200 | 2200 | 2200 | 2000 | 1200-2000 | 2400-4000 |
| Bisabolol | 2800 | 1800 | 1800 | 1800 | 2000 | 1000-1500 | 2000-3000 |

### Example 6: Liquid Compositions for Treatment of a Condition Selected from the Group Consisting of Day Function, Lack of Focus, Lack of Alertness

TABLE 6

| | Cannabinoids content (wt %) |
|---|---|
| | T20/C4, T15/C3, T10/C2, T10/C10, T5/C5, |
| | T3/C15, T1/C20, T0/C24, T0/C28 |
| Terpenes | |
| (ppm wt) | |
| α-pinene | 1000-3000 |
| B-pinene (optional) | 1000-2500 |
| Limonene | 1500-2500 |
| Terpinolene | 1500-2500 |
| Eucalyptol (optional) | 1500-2500 |
| Geraniol (optional) | 1500-2500 |
| Caryophyllene | 1500-3000 |
| Bisabolol (optional) | 500-1500 |

What is claimed is:

1. A liquid composition comprising a mixture of terpenes comprising alpha-pinene, myrcene and caryophyllene and at least one additional terpene selected from the group consisting of limonene, terpinolene, linalool, geraniol, humulene, nerolidol, bisabolol and combinations thereof; at least one cannabinoid; and an oil carrier.

2. The composition of claim 1, wherein said alpha-pinene is present at a concentration of from about 2000 to about 3800 ppm by weight of the total composition; said myrcene is present at a concentration of from about 1800 to about 2800 ppm by weight of the total composition; and said caryophyllene is present at a concentration of from about 2000 to about 3600 ppm by weight of the total composition; wherein said limonene, when present, is at a concentration of from about 1300 to about 2300 ppm by weight of the total composition; said terpinolene, when present, is at a concentration of from about 1300 ppm to about 2000 ppm by weight of the total composition; said linalool, when present, is at a concentration of from about 2000 to about 2800 ppm by weight of the total composition; said humulene, when present, is at a concentration of from about 1300 ppm to about 2000 ppm by weight of the total composition; said nerolidol, when present, is at a concentration of about 1500 ppm by weight of the total composition; wherein said bisabolol, when present is at a concentration of from about 2800 to about 3800 ppm by weight of the total composition; and said geraniol, when present, is at a concentration of from about 1800 to about 2300 ppm by weight of the total composition.

3. The composition of claim 1, wherein said at least one additional terpene comprises two additional terpenes, optionally selected from the group consisting of nerolidol and bisabolol; limonene and linalool; and humulene and bisabolol.

4. The composition of claim 1, wherein said at least one additional terpene comprises three additional terpenes, optionally selected from the group consisting of limonene, linalool and bisabolol; or geraniol, humulene and bisabolol.

5. The composition of claim 1, wherein said at least one additional terpene comprises five additional terpenes, optionally consisting of limonene, terpinolene, linalool, geraniol and bisabolol.

6. The composition of claim 1, comprising about 3800 ppm by weight alpha-pinene, about 2000 ppm by weight myrcene, about 3200 ppm by weight caryophyllene, about 1500 ppm by weight nerolidol and about 3800 ppm by weight bisabolol.

7. The composition of claim 1, comprising about 2800 ppm by weight alpha-pinene, about 2800 ppm by weight myrcene, about 2300 ppm by weight limonene, about 2800 ppm by weight linalool, and about 2800 ppm by weight caryophyllene.

8. The composition of claim 1, comprising about 2800 ppm by weight alpha-pinene, about 2300 ppm by weight myrcene, about 1300 ppm by weight limonene, about 2000 ppm by weight linalool, about 2800 ppm by weight caryophyllene and about 2800 ppm by weight bisabolol.

9. The method of claim 1, said composition comprising about 3600 ppm by weight alpha-pinene, about 1800 ppm by weight myrcene, about 2300 ppm by weight geraniol, about 3600 ppm by weight caryophyllene, from about 1300 ppm to about 2000 ppm by weight humulene and about 3300 ppm by weight bisabolol.

10. A liquid composition comprising a mixture of terpenes comprising alpha-pinene, myrcene, caryophyllene, nerolidol and bisabolol, and at least one additional terpene selected from the group consisting of ocimene, limonene, linalool, and combinations thereof; at least one cannabinoid; and an oil carrier.

11. The composition of claim 10, wherein said alpha-pinene is present at a concentration of from about 1000 to about 3000 ppm by weight of the total composition; said myrcene is present at a concentration of from about 1800 to about 5000 ppm by weight of the total composition; said caryophyllene is present at a concentration of from about 1400 to about 5000 ppm by weight of the total composition; said nerolidol is present at a concentration of from about 1200 to about 4300 ppm by weight of the total composition; said bisabolol is present at a concentration of from about 1000 to about 3000 ppm by weight of the total composition; said ocimene, when present, is at a concentration of about 1800 ppm by weight of the total composition; said limonene, when present, is at a concentration of from about 500 to about 1800 ppm by weight of the total composition.

12. The composition of claim 10, wherein said at least one additional terpene comprises two additional terpenes, optionally consisting of ocimene and linalool; or limonene and linalool.

13. The composition of claim 10, comprising about 2800 ppm by weight alpha-pinene; about 3600 ppm by weight myrcene; about 1400 ppm by weight caryophyllene; about 4300 ppm by weight nerolidol; and about 2800 ppm by weight bisabolol.

14. The composition of claim 10, comprising about 2500 ppm by weight alpha-pinene; about 3000 ppm by weight myrcene; about 1800 ppm by weight ocimene; about 1600 ppm by weight caryophyllene; about 2200 ppm by weight nerolidol; and about 1800 ppm by weight bisabolol.

15. The composition of claim 10, comprising about 2500 ppm by weight alpha-pinene; about 3000 ppm by weight myrcene; about 1800 ppm by weight linalool; about 1600 ppm by weight caryophyllene; about 2200 ppm by weight nerolidol; and about 1800 ppm by weight bisabolol.

16. The composition of claim 10, comprising about 2000 ppm by weight alpha-pinene; about 2800 ppm by weight myrcene; about 1800 ppm by weight ocimene; about 2000 ppm by weight linalool; about 1400 ppm by weight caryophyllene; about 2000 ppm by weight nerolidol; and about 2000 ppm by weight bisabolol.

17. The composition of claim 10, comprising from about 1000 to about 1500 ppm by weight alpha-pinene; from about 1800 to about 2500 ppm by weight myrcene; about 500 ppm by weight limonene; from about 500 to about 1500 ppm by weight linalool; from about 1700 to about 2500 ppm by weight caryophyllene; from about 1200 to about 2000 ppm by weight nerolidol; and about 1000 to about 1500 ppm by weight bisabolol.

18. A liquid composition comprising a mixture of terpenes consisting of alpha-pinene, limonene, terpinolene and caryophyllene, optionally comprising at least one additional terpene selected from the group consisting of beta-pinene, eucalyptol, geraniol and bisabolol, or combinations thereof; at least one cannabinoid; and an oil carrier.

19. The composition of claim 18, wherein said alpha-pinene is present at a concentration of from about 1000 to about 3000 ppm by weight of the total composition; said limonene is present at a concentration of from about 1500 to about 2500 ppm by weight of the total composition; said terpinolene is present at a concentration of from about 1500 to about 2500 ppm by weight of the total composition; said caryophyllene is present at a concentration of from about 1500 to about 3000 ppm by weight of the total composition; said beta-pinene, when present, is at a concentration of about 1000 ppm to about 2500 ppm by weight of the total composition; said eucalyptol, when present, is at a concentration of from about 1500 to about 2500 ppm by weight of the total composition; and said geraniol, when present, is at a concentration of from about 1500 to about 2500 ppm by weight of the total composition.

20. The composition of claim 18, comprising about 1000 to about 3000 ppm by weight alpha-pinene; about 1500 to about 2500 ppm by weight limonene; about 1500 to about 2500 terpinolene; and about 1500 to about 3000 ppm by weight caryophyllene.
